(19) United States
(12) Patent Application Publication
(10) Pub. No.: US 2025/0277252 A1
(43) Pub. Date: Sep. 4, 2025

(54) IDENTIFYING HYDROCARBON FIELDS USING GENOMIC DATA

(71) Applicant: SAUDI ARABIAN OIL COMPANY, Dhahran (SA)

(72) Inventors: Abdulmohsen A. Al-Humam, Dammam (SA); Mohammed A. Bataweel, Dhahran (SA); Manar M. AlAhmari, Dhahran (SA)

(73) Assignee: SAUDI ARABIAN OIL COMPANY, Dhahran (SA)

(21) Appl. No.: 18/592,322

(22) Filed: Feb. 29, 2024

(51) Int. Cl. C12Q 1/64; C12Q 1/6806; C12Q 1/6874; G16B 25/10

(52) U.S. Cl. CPC C12Q 1/64; C12Q 1/6806; C12Q 1/6874; G16B 25/10

(57) ABSTRACT

Described is a method for identifying hydrocarbon fields using genomic data. Soil samples are obtained from a geographic site, and genetic analysis is performed on the soil samples to obtain genome sequence data. Gene detection is performed on the genome sequence data to determine genes present in the soil samples. Protein sequences corresponding to the determined genes are determined and used to determine the presence of proteins involved in hydrocarbon metabolization in the soil samples.

### BACKGROUND

Subsurface hydrocarbon reserves in a reservoir may be classified as proven, probable, or possible. An appraisal of the reservoir may be performed by drilling discovery wells. Further, delineation wells may be drilled in order to identify a probable outline of the hydrocarbon reservoir. By drilling a sufficient number of delineation wells, the uncertainty in the delineation of the reservoir may be reduced to an acceptable level. The drilling of delineation wells tends to be costly, time-consuming and resource-consuming. Accordingly, it may be desirable to use other methods to identify delineations of hydrocarbon reservoirs.

Research has been performed on identifying oil/gas field size based on bacterial communities that indicate the presence of crude oil or natural gas. While the identification of bacteria has been studied for enhancing the field delineation, many communities of bacteria overlap in locations, and may not be a strong indicator of the presence of hydrocarbons.

The oil industry lacks a comprehensive description of the organisms in surface locations and near-surface locations. The diversity of microorganisms on or near the surface in hydrocarbon-rich fields may be a useful source of information regarding the microbial community genes originating from the hydrocarbon-rich fields.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a method for identifying hydrocarbon fields using genomic data. The method comprises obtaining one or more soil samples collected from a geographic site and performing a genetic analysis on the one or more soil samples to obtain genome sequence data. Gene detection is performed on the genome sequence data to determine genes present in the one or more soil samples. Protein sequences corresponding to the determined genes are obtained and used to determine presence of one or more proteins involved in hydrocarbon metabolization in the one or more soil samples.

In another aspect, one or more biomarkers for hydrocarbon metabolization are discovered using the protein sequences.

In another aspect, determining presence of the one or more proteins comprises determining the presence of one or more of cytochrome P450s, alkane hydroxylase, flavin-binding monooxygenase, and alcohol dehydrogenase.

In another aspect, absence of the one or more proteins involved in hydrocarbon metabolization in the one or more soil samples is determined.

In another aspect, the geographic site is evaluated as a potential drilling site based on the presence of proteins involved in hydrocarbon metabolization.

In another aspect, the genetic analysis comprises extracting DNA from the one or more soil samples and performing amplicon sequencing on the extracted DNA.

In another aspect, genetic analysis comprises extracting DNA from the one or more soil samples; performing a whole metagenome sequencing on the extracted DNA; and obtaining metagenome segments.

In another aspect, metagenome assembly is performed to reconstruct a metagenome from the metagenome segments.

In another aspect, the whole metagenome sequencing uses whole genome shotgun sequencing.

In another aspect, the whole metagenome sequencing uses 16S rRNA sequencing.

In another aspect, identifying the proteins comprises performing functional annotation of the determined genes.

In another aspect, the method comprises predicting whether the geographic site is a hydrocarbon bearing site based on a combination of the genome sequence data and a set of geophysical data related to the geographic site.

In another aspect, the set of geophysical data comprises at least one of seismic data, gravity data, magnetic data, electrical data, electromagnetic data, and borehole data.

In another aspect, the method comprises screening for potential drilling sites using one or more artificial intelligence algorithms.

In another aspect, the one or more artificial intelligence algorithms are selected from the group consisting of artificial neural network (ANN), logistic regression, support vector machine, naïve Bayesian classifier, Bayesian inference, adaptive boosting, decision tree learning, random forest, decision-making, K-means clustering, clustering analysis, and linear regression.

In another aspect, the method comprises using a machine learning algorithm to map the genome sequence data to the presence of hydrocarbons in the one or more soil samples.

In general, in one aspect, embodiments relate to a system for identifying hydrocarbon fields using genomic data. The system comprises one or more computer processors and a memory storing instructions, when executed, causing the one or more computer processors to perform a genetic analysis on DNA extracted from one or more soil samples to obtain genome sequence data. The system further performs gene detection on the genome sequence data to determine genes present in the one or more soil samples; determines protein sequences corresponding to the determined genes; and uses the protein sequences to determine presence of one or more proteins involved in hydrocarbon metabolization in the one or more soil samples.

In another aspect, the system causes the one or more computer processors to perform a whole metagenome sequencing on DNA extracted from the one or more soil samples and obtain metagenome segments.

In another aspect, performing the whole metagenome sequencing comprises performing one of whole genome shotgun sequencing and 16S rRNA sequencing.

In another aspect, determining the protein sequences comprises performing functional annotation of the determined genes.

In another aspect, the instructions, when executed, further cause the one or more processors to screen for potential drilling sites using one or more artificial intelligence algorithms.

In light of the structure and functions described above, embodiments of the disclosure may include respective means adapted to carry out various steps and functions defined above in accordance with one or more aspects and any one of the embodiments of one or more aspect described herein.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 shows a well environment in accordance with one or more embodiments.

FIG. 2 shows a system for identifying hydrocarbon fields in accordance with one or more embodiments.

FIG. 3 shows a flowchart for a method in accordance with one or more embodiments.

FIG. 4 shows a computer system in accordance with one or more embodiments.

FIG. 5 shows a neural network in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure include systems and methods for identifying hydrocarbon fields using genomic data. Soil samples collected from a field over a reservoir, or a suspected reservoir, may be processed using a genomic analysis to obtain genomic data. The genomic data may be used to determine a presence of microbial communities known to metabolize hydrocarbons. Such microbial communities may serve as an indicator for the presence of oil and/or gas reserves. Accordingly, potential drilling sites may be determined based on the presence of these microbial communities. The described methods and systems may be used to appraise a reservoir. In comparison to appraisals performed by drilling discovery wells and delineation wells, the method in accordance with embodiments of the disclosure is more cost effective and less time and resource consuming. A detailed description is subsequently provided.

FIG. 1 schematically shows a well environment in accordance with one or more embodiments. FIG. 1 illustrates a well environment (100) that includes a hydrocarbon reservoir (“reservoir”) (102) located in a subsurface hydrocarbon-bearing formation (104). The hydrocarbon-bearing formation (104) may include a porous or fractured rock formation that resides underground, beneath the earth's surface (“surface”) (108). The reservoir (102) may include a portion of the hydrocarbon-bearing formation (104). The hydrocarbon-bearing formation (104) and the reservoir (102) may include different layers of rock having varying characteristics, such as varying degrees of permeability, porosity, and resistivity. In the example of FIG. 1, the reservoir (102) contains oil (102A) and gas (102B), trapped under a layer (or layers) of caprock (110). While a particular example of a geological formation is shown, the hydrocarbon-bearing formation (104) may exist in any other possible geological formation, without departing from the disclosure. In the example of FIG. 1, the well environment (100) further includes a well system (106). In the case of the well system (106) being operated as a production well, the well system (106) may facilitate the extraction of hydrocarbons (or “production”) from the reservoir (102). In the case of the well system (106) being operated as an injection well, the well system (106) may be used in a tertiary recovery method to displace the produced hydrocarbons and/or to maintain the pressure profile of the reservoir (102).

The well system (106) includes a wellbore (120). The wellbore (120) may include a bored hole that extends from the surface (108) into a target zone of the hydrocarbon-bearing formation (104), such as the reservoir (102). An upper end of the wellbore (120), terminating at or near the surface (108), may be referred to as the “up-hole” end of the wellbore (120), and a lower end of the wellbore, terminating in the hydrocarbon-bearing formation (104), may be referred to as the “downhole” end of the wellbore (120). The wellbore (120) may facilitate the circulation of drilling fluids during drilling operations, the flow of hydrocarbon production (“production”) (121) (e.g., oil and gas) from the reservoir (102) to the surface (108) during production operations, the injection of substances (e.g., water) into the hydrocarbon-bearing formation (104) or the reservoir (102) during injection operations, or the communication of monitoring devices (e.g., logging tools) into the hydrocarbon-bearing formation (104) or the reservoir (102) during monitoring operations (e.g., during in situ logging operations). Additionally, the well environment (100) may include an aquifer (101) that is capable of yielding water to the reservoir (102).

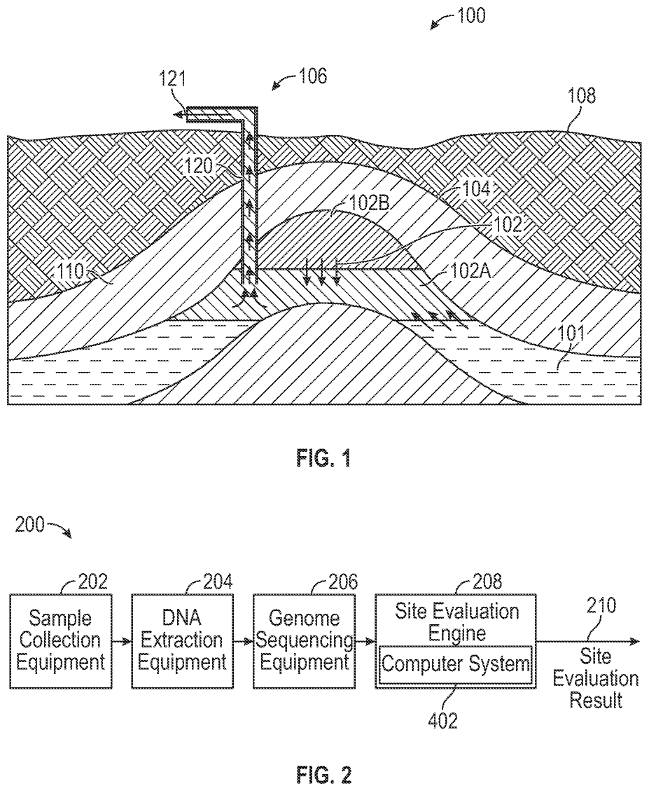

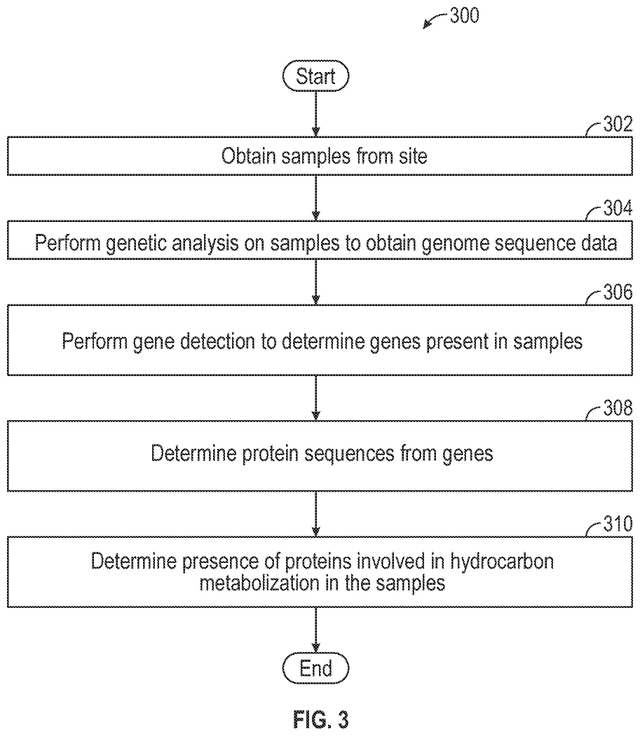

While FIG. 1 shows a well environment (100) with a wellbore (120) extending into the reservoir (102), the location and extent of the reservoir may be initially unknown. A hydrocarbon field delineation may be performed in order to determine location, geometry, and/or boundaries of the reservoir. In one or more embodiments of the disclosure, the delineation is performed using a system as shown in FIG. 2 and a method as shown in FIG. 3.

FIG. 2 shows a system (200) for identifying hydrocarbon fields in accordance with one or more embodiments. While FIG. 2 shows various configurations of hardware components and/or software components, other configurations may be used without departing from the scope of the disclosure. For example, various components in FIG. 2 may be combined to create a single component. As another example, the functionality performed by a single component may be performed by two or more components.

The system (200) may include sample collection equipment (202) for obtaining samples of interest from a geographic site, including tools to collect soil samples, containers to receive the samples, and storage elements to properly store the samples prior to analysis. The system (200) may further include DNA extraction equipment (204) for extracting DNA from the soil samples, such as chemicals necessary for DNA extraction, lab instruments, glassware, and plasticware. Non-limiting examples of lab instruments that may be utilized include a high-speed centrifuge, scale, waterbath, gel electrophoresis unit, vortex, pH meter, fluorometer, freezer, and UV transilluminator.

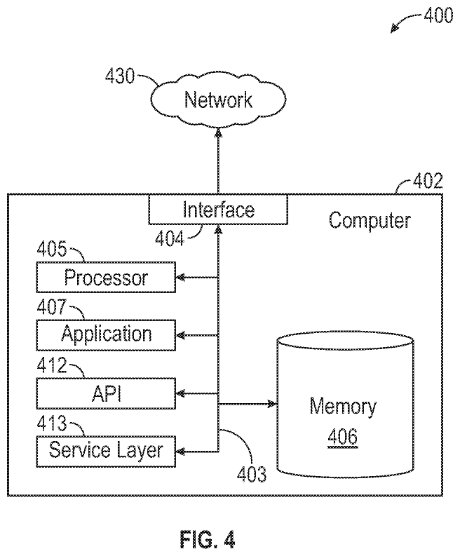

Additionally, the system (200) may include genomic sequencing equipment (206) for sequencing the extracted DNA from samples, such as a high throughput DNA sequencing machine and a polymerase chain reaction (PCR) machine. The genomic sequence data may then be analyzed with a site evaluation engine (208) including a computer system (402), such as the one depicted in FIG. 4. Based on the analysis performed by the site evaluation engine (208), a site evaluation result (210) may be generated. The site evaluation result (210) may be an indicator of whether the soil samples were collected from a potential hydrocarbon bearing site, as described in detail below.

FIG. 3 shows a flowchart in accordance with one or more embodiments. The flowchart includes operations of a method (300) for identifying hydrocarbon fields in accordance with one or more embodiments. The method may be used for oil and/or gas field delineation. Broadly speaking, embodiments of the disclosure use biomarkers developed for hydrocarbon exploration to evaluate a potential drilling site. More specifically, a sample obtained from the surface may be evaluated for the composition of its bacterial communities. In one or more embodiments, a genomic data analysis of the sample is performed to obtain genome sequences. The genome sequences may be used to predict proteins that are being generated by the organisms present in the sample obtained from the site. For example, an organism capable of metabolizing hydrocarbons, if present, or other material for energy if hydrocarbons are not present, would express certain sequences if hydrocarbons were present. These sequences, or similar sequences, may be present across different organisms that metabolize hydrocarbons, allowing the determination of the presence of hydrocarbons without requiring the identification of a specific organism. As used herein, the sequences collected are mathematically represented, for example, by numbers representing the sequence. The sequences constitute genomic data on the microorganisms present in a sample and their metabolic functions. Specific steps are subsequently described.

One or more steps in FIG. 3 may be performed by one or more components introduced in FIG. 2 and on a computer system, e.g., as shown in FIG. 4. While the various steps in FIG. 3 are presented and described sequentially, one of ordinary skill in the art will appreciate that some or all of the steps may be executed in different orders, may be combined or omitted, and some or all of the steps may be executed in parallel. Furthermore, the steps may be performed actively or passively.

In Step (302), a sample is collected from a geographical site. The site may be a geographic location to be evaluated for the presence or absence of oil and/or gas. The site may be selected analogous to how a site for drilling a discovery well of a delineation well may be selected. The site may be, for example, in a field over a reservoir. In one or more embodiments, the sample location covers a potential drilling site, and multiple samples (e.g., 5-10) are collected per site. The samples may be collected within an approximate radius of 10-50 meters from a wellhead. In a non-limiting example, 30 sites are sampled, and 5 samples are collected per site for a total of 150 samples available for analysis.

Prior to sample collection, any surface or plant litter may be removed from the desired collection location. An aseptic approach may be adhered to when handling samples to prevent sample contamination. Additionally, the samples may be collected away from any hydrocarbon contamination. Samples of soil may be collected using a tool, such as a shovel, to retrieve a dry soil surface sample at a shallow depth, such as approximately 3-5 centimeters (cm) from the surface. In one or more other embodiments, the depth of sampling is approximately 30-50 cm. Other depths outside these ranges are also possible and are dependent on the sampling site. For instance, in one or more embodiments, samples may be collected at an initial depth of one meter with subsequent collections in 20 cm intervals. The sampling depth may be recorded as metadata associated with the sample. In one or more embodiments, each soil sample contains approximately 100-200 grams of soil.

Upon collection, samples may be initially stored at −20° C. in a sterile bag or in a plastic or glass conical tube. Alternatively, the samples may be submerged in a soil preservation solution (e.g., LifeGuard Soil Preservation Solution CAT:12868-1000, QIAGEN, Germany). For transportation, samples may be stored in dry ice at approximately −78.5° C. and transported to a laboratory for analysis.

In one or more embodiments, following sample collection, genetic analysis is performed. Each sample is first processed to extract DNA from the sample. The cells in each sample may be physically or chemically lysed to release the nucleic acids from the cells in the sample. DNA may be isolated from each sample via any suitable technique known to those skilled in the art, including, but not limited to, filtration, precipitation, and/or centrifugation. Next, the isolated nucleic acids may be analyzed via spectroscopy to determine the concentration and purity of the isolated nucleic acids.

Following DNA extraction, a genomic data analysis may be performed on the sample. In one or more embodiments, a first type of DNA analysis, amplicon sequencing, may be performed on the extracted DNA. Amplicon sequencing involves analyzing genetic variation in specific genomic regions of interest. The regions of interest may range from a few genes to hundreds of genes. Amplicon sequencing uses polymerase chain reactions (PCR) to create DNA sequences, referred to as amplicons. Multiple samples may be sequenced at once using amplicon sequencing.

In one or more other embodiments, the genomic data analysis is based on the metagenome of the sample, where all DNA is sequenced rather than just the 16S or 18S gene. A metagenome is the genome contained within an environmental sample which may include multiple organisms. Analysis of the metagenome may indicate the presence of different organisms, as well as identify organisms that use hydrocarbons for energy in addition to other sources of energy. For instance, detection of bacteria known to metabolize hydrocarbons, such as the genera Oleispira, Oleiphilus, Thalassolituus, Alcanivorax, and Cycloclasticu, may be utilized as an indicator of a hydrocarbon field. In addition, correlating surface microorganisms with sub-surface microorganisms from cuttings may provide additional information.

The metagenomic analysis is a second type of DNA analysis that may be performed in order to recover and completely sequence the genetic material of the microbial communities in the sample. In one or more embodiments, whole metagenome sequencing is performed in order to obtain genomic data of the genetic material across the entire sample, rather than being limited to certain taxa. In one or more embodiments, based on the obtained genomic data, corresponding proteins may be identified. The identified proteins may be reviewed to determine whether proteins that are indicators of hydrocarbon presence are found in the sample. Such proteins may be proteins involved in, or capable of, metabolizing or processing hydrocarbons, and are, thus, considered indicators for the presence of oil and/or gas. Both amplicon sequencing and metagenome sequencing allow identification of protein-coding genes that are associated with the presence of hydrocarbons.

The metagenomic analysis involved is subsequently described in Steps (304) through (310) of FIG. 3. In Step (304), a metagenome sequencing may be performed on the sample to obtain genome sequence data. The metagenome sequencing may produce metagenome segments. The execution of Step (304) may rely on metagenome sequencing using, for example, 16S rRNA sequencing, whole-genome shotgun (WGS) sequencing, or both. The analysis of the rRNA allows for the taxonomic identification of microorganisms, such as bacterial communities present in a sample. Alternatively, marker gene studies may be used for high-throughput sequencing. Common marker genes used in microbial ecology are the 16S rRNA gene (archaea and bacteria), the internal transcribed spacer (ITS) region (fungi), and the 18S rRNA (eukaryotes).

As used herein, shotgun metagenomics analyzes samples for genomic material from thousands of organisms in parallel. This approach provides insight into community biodiversity and functions. Further, shotgun sequencing allows for the detection of low abundance members of microbial communities. Shotgun metagenomics analyzes all genomic DNA in a sample rather than a specific region of DNA, as in 16S rRNA sequencing. Thus, using shotgun metagenomic sequencing, simultaneous identification of bacteria, fungi, viruses, and other microorganisms is possible.

In Step (306), a metagenome assembly may be performed to reconstruct the metagenome from the metagenome segments. The metagenome assembly is performed by sequence assembly algorithms able to reconstruct genes and organisms from complex mixtures as they may be present in the sample. Assembly involves reconstructing in silico the original genome sequence from smaller fragments. The assembly may be performed by joining sequenced fragments to generate a set of DNA segments, or sequences that overlap in a manner that provides a contiguous representation of a genomic region. The technique does not use a reference genome. Alternatively, assembly may be carried out using previously sequenced, closely related organisms as a reference to guide the assembly.

In Step (308), a gene detection is performed to determine the genes associated with the metagenome. The gene detection may be performed using correlation or lookup operations involving databases known to one skilled in the art. Some of the determined genes may encode proteins. Accordingly, protein sequences may be determined. All protein-coding genes in the sample may be screened. Then, the functions of the proteins may be predicted using artificial intelligence (AI) methods for protein function prediction. In this manner, detection of known and new (previously unknown) biomarkers for the presence of hydrocarbons may be identified to provide a functional view of the microbes in the soil samples. Discovering novel biomarkers for the presence of hydrocarbons may then be useful in future predictions of drilling sites.

Referring to FIG. 3, in Step (310), a functional annotation of the genes may be performed to identify proteins involved in hydrocarbon metabolization. Functional annotation is used to relate biological information to sequences of genes or proteins and then annotate (or label) the genes or proteins. In one or more embodiments, the basic local alignment search tool (BLAST) may be used to compare gene or protein sequences to sequence databases and calculate the statistical significance of the comparison. Additionally, the functional annotation may be performed using tools, such as InterproScan and/or DeepGOPlus, which allow the submission of a nucleotide or protein sequences to be functionally characterized. Matches are then calculated, for example, against database entries for proteins known to be involved in hydrocarbon metabolization, such as cytochrome P450s, alkane hydroxylase, flavin-binding monooxygenase, and alcohol dehydrogenase. Other methods for predicting protein function may be used, without departing from the disclosure. In one or more embodiments, any detectable amount of proteins known to be involved in hydrocarbon metabolism in the soil samples is a sufficient indicator for a drilling site. Alternatively, a threshold amount for a particular protein (or proteins) may be set as an indicator. Additionally, the presence of proteins known to not be found in locations having oil may be used as negative indicators.

The identification and characterization of genomic data described above may be used in a computational tool based on artificial intelligence (AI) algorithms for identifying and screening potentially successful drilling sites. In one or more embodiments, the genomic information may be used to develop a computational tool that implements the taxonomic and functional microbial information to identify successful hydrocarbon bearing sites. The genomic data may be combined with other geological and geophysical data. For instance, a set of geophysical data may include one more of seismic data, gravity data, magnetic data, electrical data, electromagnetic data, and borehole data. The combination of genomic data with other data types may enhance the accuracy of predicting and locating hydrocarbon bearing sites, thereby lowering the costs of finding the sites.

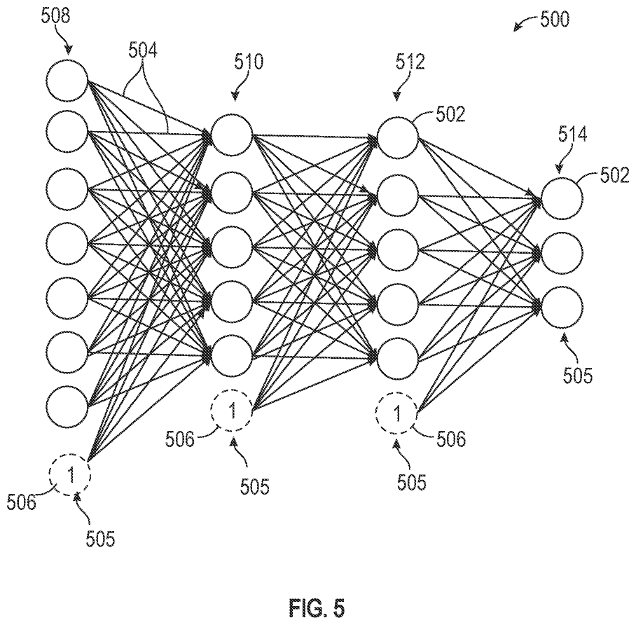

In one or more embodiments, artificial intelligence (AI) algorithms, or machine learning algorithms, are trained to map the genes and proteins in microbes to the presence or absence of oil and/or gas in soil samples. In other words, protein-coding genes in the sample are screened, and the functions of these proteins are predicted using AI methods for protein function prediction. This allows for detection of known as well as novel markers for presence of hydrocarbons. Non-limiting examples of machine learning/AI algorithms that may be implemented in the method described herein include artificial neural network (ANN), logistic regression, support vector machine, naïve Bayesian classifier, Bayesian inference, adaptive boosting (Adaboost), decision tree learning, random forest, decision-making, K-means clustering, clustering analysis, and linear regression. An example of a machine learning model (e.g., a neural network) is shown in FIG. 5 and described below. From the soil samples, thousands of biomarkers (e.g., DNA fingerprints) may be obtained, with only a few qualified to serve as key indicators of a positive or negative presence of oil. The biomarkers may then be input to an AI computer model, which generates a “sweet spot” map.

Advantageously, embodiments disclosed herein provide a “functional” view of the microbes in the soil, rather than just a lookup for known markers. Embodiments described herein enable discovery of biomarkers, and not just screen for the presence of known ones.

Embodiments may be implemented on a computer system. FIG. 4 is a block diagram of a computer system (402) used to provide computational functionalities associated with described algorithms, methods, functions, processes, flows, and procedures as described in the instant disclosure, according to an implementation. The illustrated computer system (402) is intended to encompass any computing device such as a high-performance computing (HPC) device, a server, desktop computer, laptop/notebook computer, wireless data port, smart phone, personal data assistant (PDA), tablet computing device, one or more computer processors within these devices, or any other suitable processing device, including both physical or virtual instances (or both) of the computing device. Additionally, the computer system (402) may include a computer that includes an input device, such as a keypad, keyboard, touch screen, or other device that can accept user information, and an output device that conveys information associated with the operation of the computer system (402), including digital data, visual, or audio information (or a combination of information), or a GUI.

The computer system (402) can serve in a role as a client, network component, a server, a database or other persistency, or any other component (or a combination of roles) of a computer system for performing the subject matter described in the instant disclosure. The illustrated computer system (402) is communicably coupled with a network (430). In some implementations, one or more components of the computer system (402) may be configured to operate within environments, including cloud-computing-based, local, global, or other environment (or a combination of environments).

At a high level, the computer system (402) is an electronic computing device operable to receive, transmit, process, store, or manage data and information associated with the described subject matter. According to some implementations, the computer system (402) may also include or be communicably coupled with an application server, e-mail server, web server, caching server, streaming data server, business intelligence (BI) server, or other server (or a combination of servers).

The computer system (402) can receive requests over network (430) from a client application (for example, executing on another computer system (402)) and responding to the received requests by processing the said requests in an appropriate software application. In addition, requests may also be sent to the computer system (402) from internal users (for example, from a command console or by other appropriate access method), external or third-parties, other automated applications, as well as any other appropriate entities, individuals, systems, or computers.

Each of the components of the computer system (402) can communicate using a system bus (403). In some implementations, any or all of the components of the computer system (402), both hardware or software (or a combination of hardware and software), may interface with each other or the interface (404) (or a combination of both) over the system bus (403) using an application programming interface (API) (412) or a service layer (413) (or a combination of the API (412) and service layer (413). The API (412) may include specifications for routines, data structures, and object classes. The API (412) may be either computer-language independent or dependent and refer to a complete interface, a single function, or even a set of APIs. The service layer (413) provides software services to the computer system (402) or other components (whether or not illustrated) that are communicably coupled to the computer system (402). The functionality of the computer system (402) may be accessible for all service consumers using this service layer. Software services, such as those provided by the service layer (413), provide reusable, defined business functionalities through a defined interface. For example, the interface may be software written in JAVA, C++, or other suitable language providing data in extensible markup language (XML) format or other suitable format. While illustrated as an integrated component of the computer system (402), alternative implementations may illustrate the API (412) or the service layer (413) as stand-alone components in relation to other components of the computer system (402) or other components (whether or not illustrated) that are communicably coupled to the computer system (402). Moreover, any or all parts of the API (412) or the service layer (413) may be implemented as child or sub-modules of another software module, enterprise application, or hardware module without departing from the scope of this disclosure.

The computer system (402) includes an interface (404). Although illustrated as a single interface (404) in FIG. 4, two or more interfaces (404) may be used according to particular needs, desires, or particular implementations of the computer system (402). The interface (404) is used by the computer system (402) for communicating with other systems in a distributed environment that are connected to the network (430). Generally, the interface (404 includes logic encoded in software or hardware (or a combination of software and hardware) and operable to communicate with the network (430). More specifically, the interface (404) may include software supporting one or more communication protocols associated with communications such that the network (430) or interface's hardware is operable to communicate physical signals within and outside of the illustrated computer system (402).

The computer system (402) includes at least one computer processor (405). Although illustrated as a single computer processor (405) in FIG. 4, two or more processors may be used according to particular needs, desires, or particular implementations of the computer system (402). Generally, the computer processor (405) executes instructions and manipulates data to perform the operations of the computer system (402) and any algorithms, methods, functions, processes, flows, and procedures as described in the instant disclosure.

The computer system (402) also includes a memory (406) that holds data for the computer system (402) or other components (or a combination of both) that can be connected to the network (430). For example, memory (406) can be a database storing data consistent with this disclosure. The memory (406) may store instructions that, when executed, cause one or more computer processors to perform multiple computer-implemented operations. Although illustrated as a single memory (406) in FIG. 4, two or more memories may be used according to particular needs, desires, or particular implementations of the computer system (402) and the described functionality. While memory (406) is illustrated as an integral component of the computer system (402), in alternative implementations, memory (406) can be external to the computer system (402).

The application (407) is an algorithmic software engine providing functionality according to particular needs, desires, or particular implementations of the computer system (402), particularly with respect to functionality described in this disclosure. For example, application (407) can serve as one or more components, modules, applications, etc. Further, although illustrated as a single application (407), the application (407) may be implemented as multiple applications (407) on the computer system (402). In addition, although illustrated as integral to the computer system (402), in alternative implementations, the application (407) can be external to the computer system (402).

There may be any number of computer systems (402) associated with, or external to, a computer system containing computer system (402), each computer system (402) communicating over network (430). Further, the term “client,” “user,” and other appropriate terminology may be used interchangeably as appropriate without departing from the scope of this disclosure. Moreover, this disclosure contemplates that many users may use one computer system (402), or that one user may use multiple computer systems (402).

In some embodiments, the computer system (402) is implemented as part of a cloud computing system. For example, a cloud computing system may include one or more remote servers along with various other cloud components, such as cloud storage units and edge servers. In particular, a cloud computing system may perform one or more computing operations without direct active management by a user device or local computer system. As such, a cloud computing system may have different functions distributed over multiple locations from a central server, which may be performed using one or more Internet connections. More specifically, a cloud computing system may operate according to one or more service models, such as infrastructure as a service (IaaS), platform as a service (PaaS), software as a service (SaaS), mobile “backend” as a service (MBaaS), serverless computing, artificial intelligence (AI) as a service (AIaaS), and/or function as a service (FaaS).

As noted above in the discussion of FIG. 3, machine learning algorithms implemented using machine learning models may be used to map the genes and proteins in microbes to the presence or absence of oil and/or gas in soil samples. The input into the ML model is protein-coding genes in the sample, and the output is the functions of these proteins, which are predicted using the ML model for protein function prediction. A diagram of an exemplary machine learning model, e.g., a neural network (500) as may be implemented in the method described herein, is shown in FIG. 5. At a high level, a neural network (500) may be graphically depicted as being composed of nodes (502), where here any circle represents a node, and edges (504), shown here as directed lines. The nodes (502) may be grouped to form layers (505). FIG. 5 displays four layers (508, 510, 512, 514) of nodes (502) where the nodes (502) are grouped into columns, however, the grouping need not be as shown in FIG. 5. The edges (504) connect the nodes (502). Edges (504) may connect, or not connect, to any node(s) (502) regardless of which layer (505) the node(s) (502) is in. That is, the nodes (502) may be sparsely and residually connected. A neural network (500) will have at least two layers (505), where the first layer (508) is considered the “input layer” and the last layer (514) is the “output layer.” Any intermediate layer (510, 512) is usually described as a “hidden layer.” A neural network (500) may have zero or more hidden layers (510, 512) and a neural network (500) with at least one hidden layer (510, 512) may be described a “deep” neural network or a “deep learning method.” In general, a neural network (500) may have more than one node (502) in the output layer (514). In this case the neural network (500) may be referred to as a “multi-target” or “multi-output” network.

When the neural network (500) receives a network input, the network input is propagated through the network according to the activation functions and incoming node (502) values and edge (504) values to compute a value for each node (502). That is, the numerical value for each node (502) may change for each received input. Occasionally, nodes (502) are assigned fixed numerical values, such as the value of 1, that are not affected by the input or altered according to edge (504) values and activation functions. Fixed nodes (502) are often referred to as “biases” or “bias nodes” (506), displayed in FIG. 5 with a dashed circle.

In some implementations, the neural network (500) may contain specialized layers (505), such as a normalization layer, a regularization layer (e.g. dropout layer), and a concatenation layer. One skilled in the art will appreciate that these alterations do not exceed the scope of this disclosure.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A method for identifying hydrocarbon fields using genomic data, the method comprising:
obtaining one or more soil samples collected from a geographic site;
performing a genetic analysis on the one or more soil samples to obtain genome sequence data;
performing gene detection on the genome sequence data to determine genes present in the one or more soil samples;
determining protein sequences corresponding to the determined genes; and
using the protein sequences, determining presence of one or more proteins involved in hydrocarbon metabolization in the one or more soil samples.

2. The method of claim 1, wherein determining presence of the one or more proteins comprises determining the presence of one or more of cytochrome P450s, alkane hydroxylase, flavin-binding monooxygenase, and alcohol dehydrogenase.

3. The method of claim 1, further comprising discovering one or more biomarkers for hydrocarbon metabolization using the protein sequences.

4. The method of claim 1, further comprising determining absence of the one or more proteins involved in hydrocarbon metabolization in the one or more soil samples.

5. The method of claim 1, further comprising evaluating the geographic site as a potential drilling site based on the presence of proteins involved in hydrocarbon metabolization.

6. The method of claim 1, wherein performing the genetic analysis comprises:
extracting DNA from the one or more soil samples; and
performing amplicon sequencing on the extracted DNA.

7. The method of claim 1, wherein performing the genetic analysis comprises:
extracting DNA from the one or more soil samples;
performing a whole metagenome sequencing on the extracted DNA; and
obtaining metagenome segments.

8. The method of claim 7, further comprising performing metagenome assembly to reconstruct a metagenome from the metagenome segments.

9. The method of claim 7, wherein the whole metagenome sequencing uses whole-genome shotgun sequencing.

10. The method of claim 7, wherein the whole metagenome sequencing uses 16S rRNA sequencing.

11. The method of claim 1, wherein determining the protein sequences comprises performing functional annotation of the determined genes.

12. The method of claim 1, further comprising predicting whether the geographic site is a hydrocarbon bearing site based on a combination of the genome sequence data and a set of geophysical data related to the geographic site.

13. The method of claim 12, wherein the set of geophysical data comprises at least one of seismic data, gravity data, magnetic data, electrical data, electromagnetic data, and borehole data.

14. The method of claim 1, further comprising screening for potential drilling sites using one or more artificial intelligence algorithms.

15. The method of claim 14, wherein the one or more artificial intelligence algorithms are selected from the group consisting of artificial neural network (ANN), logistic regression, support vector machine, naïve Bayesian classifier, Bayesian inference, adaptive boosting, decision tree learning, random forest, decision-making, K-means clustering, clustering analysis, and linear regression.

16. The method of claim 1, further comprising using a machine learning algorithm to map the genome sequence data to the presence of hydrocarbons in the one or more soil samples.

17. A system for identifying hydrocarbon fields using genomic data, comprising:
one or more computer processors; and
a memory storing instructions, when executed, causing the one or more computer processors to:
perform a genetic analysis on DNA extracted from one or more soil samples collected from a geographic site to obtain genome sequence data;
perform gene detection on the genome sequence data to determine genes present in the one or more soil samples;
determine protein sequences corresponding to the determined genes; and
using the protein sequences, determining presence of one or more proteins involved in hydrocarbon metabolization in the one or more soil samples.

18. The system of claim 17, the instructions, when executed, further causing the one or more computer processors to:
perform a whole metagenome sequencing on DNA extracted from the one or more soil samples; and
obtain metagenome segments.

19. The system of claim 18, wherein performing the whole metagenome sequencing comprises performing one of whole-genome shotgun sequencing and 16S rRNA sequencing.

20. The system of claim 17, wherein determining the protein sequences comprises performing functional annotation of the determined genes.
